(19) United States
(12) Patent Application Publication
(10) Pub. No.: US 2016/0143314 A1
(43) Pub. Date: May 26, 2016

(54) CONFECTION DELIVERY APPARATUS

(71) Applicant: Frank Clark, Albuquerque, NM (US)

(72) Inventor: Frank Clark, Albuquerque, NM (US)

(21) Appl. No.: 14/642,599

(22) Filed: Mar. 9, 2015

Related U.S. Application Data
(63) Continuation-in-part of application No. 14/550,786, filed on Nov. 21, 2014.

(51) Int. Cl. A23G 3/54; A61Q 11/00; A61K 8/11

(52) U.S. Cl. CPC A23G 3/54; A61K 8/11; A61Q 11/00; A23G 2220/20

(57) ABSTRACT

A confection delivery apparatus includes a chewable set of spaced apart baffles formed from a pliable and non-resilient material. The baffles are connected perpendicular to an anchor. A joint connects to the anchor. A stem connects to the joint. The baffles curve toward the stem. The space between the baffles include a confection. An edible wax coating adheres to the baffles and the confection.

### CROSS-REFERENCE TO RELATED APPLICATIONS

This application is a continuation-in-part of U.S. Ser. No. 14/550,786, filed on Nov. 21, 2014, the entire contents of which are incorporated herein by reference.

### TECHNICAL FIELD

The present invention relates generally to a confection delivery apparatus. More particularly, the present invention relates to a confection delivery apparatus having a stem and a chewable portion.

### BACKGROUND

Confections such as sweets and candies are widely consumed and enjoyed. However, the flavor rush that is experienced when a user eats candy may be fleeting if the candy is small or if the user eats the candy quickly. Candies such as a chocolate covered candy and soft candy may also be messy to handle and are also difficult to pair with other consumable items. For example, little, if any, thought has been given to physically pairing a confection with a drink in a manner that improves usability, eliminates mess, provides aeration, and simultaneously extends and enhances the flavor rush.

A chewable toothbrush is known (U.S. Pat. No. 6,602,013) that releases a wetting material such as toothpaste and mouthwash onto a set of bristles upon application of a chewing force, but does not contemplate extending the flavor rush of a confection with aeration in a new and unconventional manner.

### SUMMARY

A confection delivery apparatus is provided that enhances the flavor of a confection and extends a flavor rush to creates a new and unique way to enjoy treats and other candied products. The confection delivery apparatus as described herein also eliminates the mess from soft candy treats, pairs easily with a drink, and even provides cooling for a hot drink.

In some embodiments of the invention, a confection delivery apparatus includes a chewable set of spaced apart baffles formed from a pliable and non-resilient material. The baffles are connected perpendicular to an anchor. A joint connects to the anchor. A stem connects to the joint. The baffles curve toward the stem. The space between the baffles include a confection. An edible wax coating adheres to the baffles and the confection.

Optionally, the anchor is thinner than the joint. The baffles may be thinner than the stem. The baffles may define at least one of a smooth, rough and ribbed surface. The baffles may be formed by injection molding. The baffles may define at least one of an ellipsoid and a polygon shape. The baffles may vary in diameter. The stem may define at least one hanger. The edible wax coating may temporarily seal the confection within the baffles.

In another embodiments, a confection delivery apparatus includes a chewable set of spaced apart branches formed from a pliable and non-resilient material. The branches are connected to an anchor. A joint connects to the anchor. A stem connects to the joint. The branches curve toward the stem. The space between the branches include a confection. An edible wax coating is adhered to the branches and the confection.

Optionally, the anchor may be thinner than the stem. The branches may be thin collapsible brush strands. The branches and the anchor may be formed by injection molding. The branches may vary in length. A first portion of the branches may be covered by a first confection and a second portion of the branches may be covered by a second confection. The edible wax may be laced with a flavoring. The stem may define a first end contacting the joint, a second end opposite the first end. The second end may define a flat handle. The confection may include any of a breath mint, dessert mint, banana cream pie, and coconut cream pie. A cup of liquid is attachable to the stem of the apparatus.

In some embodiments, a method of forming a confection delivery apparatus includes the steps of cooling a chewable set of spaced apart baffles formed from a pliable and non-resilient material. The baffles are connected perpendicular to an anchor. A joint is connected to the anchor. A stem is connected to the joint. The baffles curve toward the stem. A confection is loaded in the baffles. The baffles are coated with a melted confection. The melted confection is cooled so as to form a melted confection coating adhered to the baffles. The melted confection coating of the confection delivery apparatus is coated with a melted edible wax. The melted edible wax is cooled so as to form an edible wax coating adhered to the melted confection coating. The confection is forced through the baffles in response to an external force and remains compressed in response to removal of the external force.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying non-limiting drawings of which:

FIG. 1 is a schematic view of a portion of a confection delivery apparatus, in accordance with many embodiments.

FIG. 2 is a schematic view of a confection delivery apparatus, in accordance with many embodiments.

FIG. 3 is a cross-sectional view of a confection delivery apparatus, in accordance with many embodiments.

FIG. 4 is a schematic view of a portion of a confection delivery apparatus, in accordance with many embodiments.

FIG. 5 is a schematic view of a confection delivery apparatus, in accordance with many embodiments.

FIG. 6 is a schematic view of a confection delivery apparatus, in accordance with many embodiments.

FIG. 7 is a cross-sectional view of a confection delivery apparatus and a cup, in accordance with many embodiments.

FIG. 8 is a cross-sectional view of a confection delivery apparatus and a cup, in accordance with many embodiments.

FIG. 9 is a cross-sectional view of a confection delivery apparatus and a cup, in accordance with many embodiments.

FIG. 10 is a cross-sectional view of a confection delivery apparatus, in accordance with many embodiments.

FIG. 11 is a detailed schematic view of a confection delivery apparatus, in accordance with many embodiments.

FIG. 12 is a schematic view of a confection delivery apparatus, in accordance with many embodiments.

FIG. 13 is a cross-sectional view of a confection delivery apparatus and a cup, in accordance with many embodiments.

FIG. 14 is a schematic view of a confection delivery apparatus, in accordance with many embodiments.

FIG. 15 provides schematic views of a confection delivery apparatus, in accordance with many embodiments.

FIG. 16 provides schematic views of a confection delivery apparatus, in accordance with many embodiments.

FIG. 17 provides schematic views of a confection delivery apparatus, in accordance with many embodiments.

FIG. 18 provides perspective views of a confection delivery apparatus, in accordance with many embodiments.

FIG. 19 provides schematic views of a confection delivery apparatus, in accordance with many embodiments.

FIG. 20 provides schematic views of a confection delivery apparatus, in accordance with many embodiments.

### DETAILED DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments.

The term confection used herein refers to a consumable food product including candies, sweets, a sweet preparation combined with another food, and the like. For example, a confection includes breath freshening products, chocolate covered mint candies, chocolate covered espresso and regular coffee beans, etc.

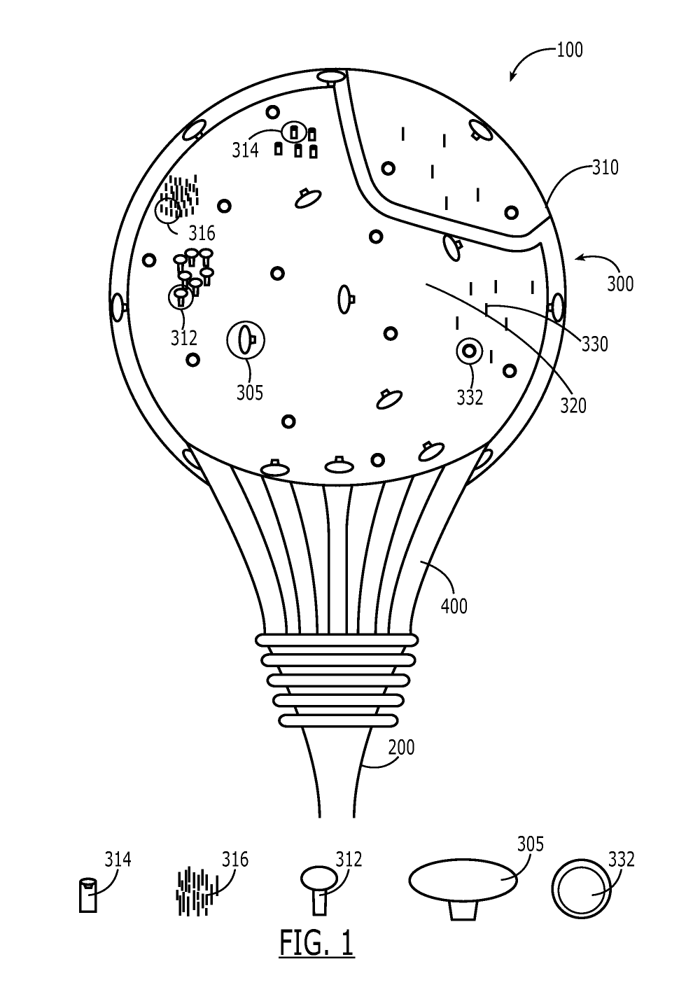

FIG. 1 is a schematic view of a portion of the confection delivery apparatus, in accordance with many embodiments. The example embodiment described herein is suitable as a single-use, disposable confection delivery apparatus 100. Alternatively, apparatus 100 can be configured for multiple uses. Apparatus 100 generally includes a stem 200 and a container 300 and a joint 400 for connecting the stem 200 to the container 300. The joint 400 may be reinforced to anchor the stem 200 to the container 300 and includes a flex joint 410. The length and thickness of the stem 200 may vary with particular design requirements, and may be a round shaft of approximately 3″-6″ in length.

A second end of the stem 200 will be described in detail later and is provided opposite the container 300. The stem 200 may be formed of the same material as the container 300. A first end of the stem 200 is connected to the joint 400 and flex joint 410, which may include ribs that allow the stem 200 to flex in a plurality of directions and angles with respect to the container 300. The flex joint 410 is bendable. From the second end of the stem 200, the joint 400 flares out and become thicker to attach securely to the container 300. The joint 400 broadens and attaches to the container 300 in this manner in order to withstand any external force that is placed on the container 300 and/or the joint 400, such as a biting or chewing force.

In particular, the container 300 is preferably formed from a pliable material that compresses when chewed. The container initially holds an uncompressed shape and compresses upon itself in response to the application of an external force (such as a chewing force) and retains the compressed shape in response to removal of the external force. Container 300 may be formed from a non-resilient material that does not return to its original shape when compressed by an external force. The joint 400 may be formed of a resilient material having elastic memory that allows the joint 400 to return to its original shape. The container 300 and joint 400 may be formed from any suitable plastic, rubber, nylon, composite or other material. However, the hardness, resiliency, elasticity, tensile strength and other physical properties of the container 300 and joint 400 may be selected in accordance with the confection and the application of a large external compressing force such as a bite force. The joint 400 may be reinforced.

The outer surface of the container 300 may include at least one wax anchor 305 that may be generally solid and spherical in shape to promote adhesion of edible wax to the container surface. The wax anchor may be formed of the same material as the container 300. In particular, wax anchor 305 protrudes from the container 300 and includes an oval end while wax anchor 312 has a rounded end. A hollow wax anchor 314 protrudes from the container 300. The wax anchor 316 includes a plurality of short rounded bristles. The wax anchors may be uniformly shaped. The container 300 may include a ribbed surface 320. Seep slits 330 and seep holes 332 are conduits that allow the contents of the container 300 to seep out of the container 300. The seep holes 332 may be 1/16″-¼″ in diameter, for example.

Any texture, protrusion or hole configuration may be provided as a wax anchor that suitably aids in edible wax adhesion to the container 300. The different wax anchor configurations may be combined as necessary to promote edible wax adhesion. The wax anchor structures described herein may be attached to container 300 in any suitable manner. For example, the wax anchor may be molded into container 300, inserted and bonded into holes formed in the container 300, and the like. The anchors may be located in a random manner or in a specified pattern suited to the shape, size and application of the apparatus 100.

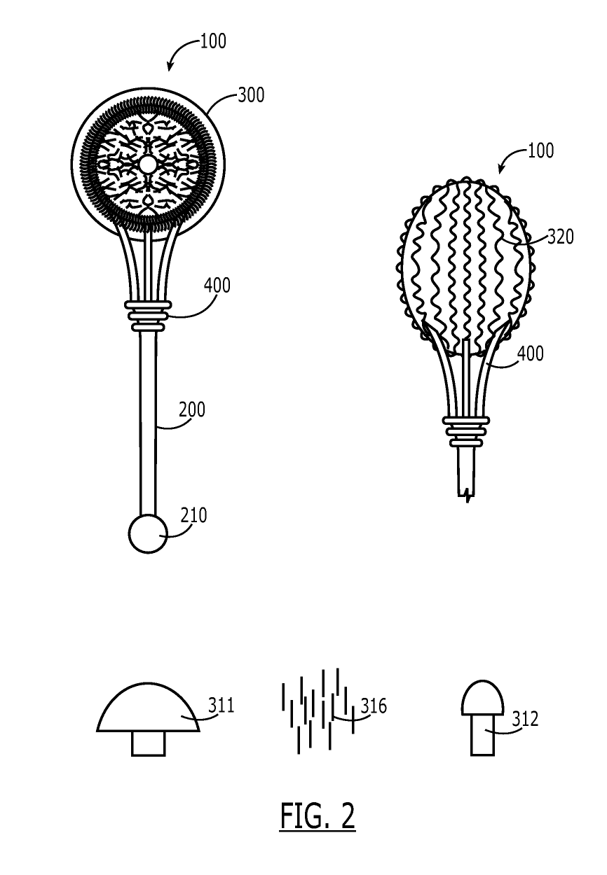

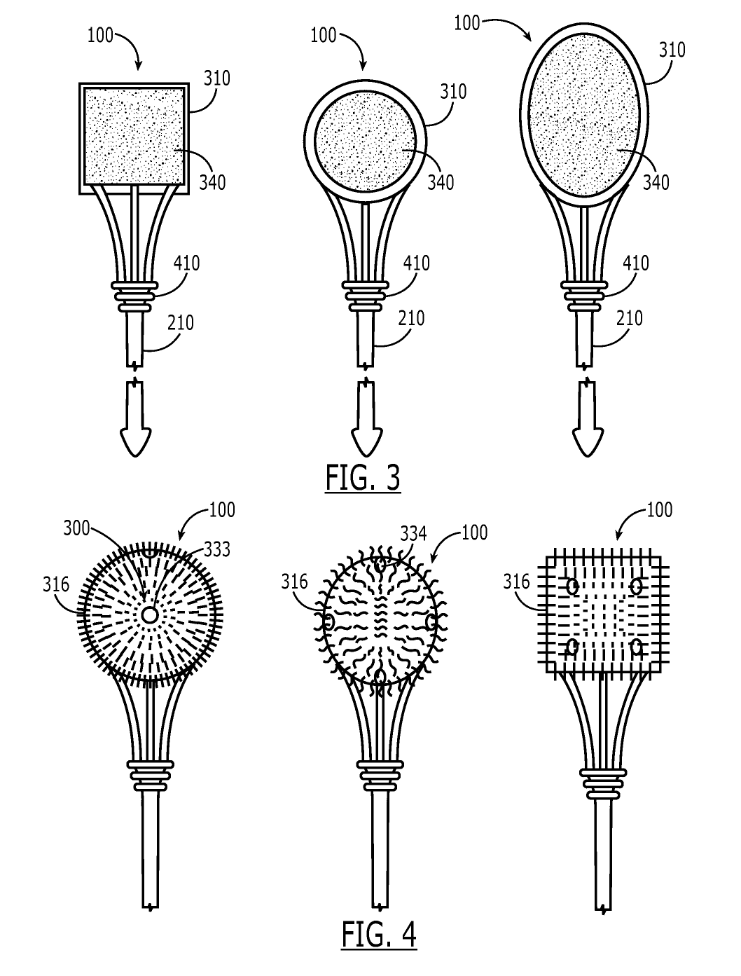

FIG. 2 is a schematic view of the confection delivery apparatus, in accordance with many embodiments. An apparatus 100 includes a stem 200 including a first end 210, a container 300 and joint 400. In FIG. 2, the container 300 includes a plurality of short bristles as the wax anchor. The wax anchors may include one or more of bristles 316, a short stem with a round end 312 and a short stem with a mushroom shaped end cap 311. The surface of the container 300 may include a rough surface 320 for the edible wax to adhere and cling. A first end 210 of the stem 200 may include a rounded end or curved point such as a curved toothpick (FIG. 3). Stem 200 may be formed from the same pliant and non-resilient material as the container 300, a resilient material like that used for joint 400, or it may be formed from any suitable material such as plastic. Stem 200 may be rigid, adjustable, or flexible, depending upon the particular design. Although not a requirement of the present invention, the apparatus 100 may be formed as an integral unit via injection molding, ensuring no separation between elements of the apparatus 100 or any suitable manufacturing process. The components of the apparatus 100 may be all of the same material, but varied in strength, flexibility and resiliency based upon a thickness and/or shape of the component. For example, a thinner portion of the stem 200 may be adjustable and flexible but a thicker portion may be inflexible and not subject to compression by the application of an external bite force. Similarly, the container 300 may be thinner than the stem 200 so as to allow the container 300 to compress and remain collapsed in response to an application of external force.

FIG. 3 is a cross-sectional view of the confection delivery apparatus 100, in accordance with many embodiments. The containers 300 may be formed in a plurality of shapes such as round, oval, oblong, square, star, diamond, and the like, so long as a hollow cavity 340 is formed within. The cavity 340 of the container 300 is filled with a confection and surrounded by an edible wax cocoon 310 over the container 300. The cavity 340 may be filled with a plurality of different confections, such as at least one to four confections. Filling the cavity 340 with multiple confections produces meltable flavors that complement each other to enhance the user experience. An edible wax cocoon 310 is provided surrounding each of the different container 300 shapes. The outer surface of the container 300 may be smooth, rough or ribbed based on the contents of the cavity 340. After the confection(s) are inserted into the container 300, the container 300 is submerged in an edible hot wax that forms the cocoon 310. The type of edible wax to form the cocoon 310 is based on the contents of the cavity 340. The hardness of the edible wax and temperature of submersion will also vary according to the contents of the cavity 340 and application of the apparatus 100. The edible wax is formed on and between the wax anchors 305 and help retain the edible wax cocoon 310 during manufacturing, shipping and storage.

For instance, a high temperature edible wax will be used when the melting point requires a higher temperature such as a chocolate covered coffee bean to be submerged in a cup of hot coffee, whereas simple room temperature exposure would require a low temperature edible wax to form the edible wax cocoon 310. After the container 300 is submerged in hot edible wax and quickly cooled, the edible wax cocoon 310 forms around the entirety of the container 300 to seal and form an airtight seal. The wax anchors on the outer surface of the container 300 are provided to form additional surface area for the edible wax to bond strongly to the container 300. Furthermore, prior to forming the wax coating with the edible wax, the edible wax itself may be enhanced by mixing with a flavor. The flavored edible wax is then melted and cooled over the container 300 to form a flavored wax cocoon 310 that will complement the contents of the cavity 340.

The hardness and chewable consistency of the edible wax may also be selected based on the contents of the container 300 so that the chewiness of the wax complements the chewiness of the confection. This allows th contents of the container 300 to easily mix with the edible wax cocoon. In this manner, as the edible wax cocoon is chewed and mixed with the juices of the mouth, flavor and enjoyment is enhanced.

Cavity 340 is suitably configured to hold a confection that is released when an external force is applied to container 300. The apparatus 100 may be specifically sized for use by adults or children, i.e., the size of wax anchors 305 and cavity 340 can be relatively small or relatively large depending upon the intended use. For example, cavity 340 may be sized to contain any volume of material, e.g., between 2 to 10 milliliters. When a user bites down on apparatus 100, the edible wax cocoon 310 and the container 300 are compressed, thus crushing the contents of the cavity 340 and forcing the contents through the seep holes 332 or slits 330 in the container 300, thereby mixing with the chewed edible wax cocoon 310.

Seep holes 332 and seep slits 330 are suitably configured to release the confection contained in cavity 340. When container 300 is compressed, the substance contained in cavity 340 squeezes through seep holes 332 or seep slits 330. Of course, the size, shape, and arrangement of seep holes 332 and seep slits 330 can vary according to the particular design and application of apparatus 100. When apparatus 100 is being chewed, the confection exits cavity 340 and comes into contact with the edible wax cocoon 310 and eventually into contact with the user's mouth. The edible wax cocoon 310 is relatively hard prior to use and becomes relatively soft during use. The wax cocoon may be infused with a flavored substance adding further flavor enhancement to complement the apparatus 100. When chewed, the edible wax mixes with the confection released from cavity 340, and is warmed by the user's mouth, the edible wax becomes soft and mixes with the confection and mouth juices. In this manner, the apparatus 100 extends the flavor rush of the confection.

The released confection is trapped in the edible wax cocoon 310 upon application of external force and is folded over itself, simultaneously mixing with the wetness of the mouth, thereby combining flavored contents with folded edible wax and mouth juices to extend the flavor rush. The chewed edible wax disperses the trapped flavor contents continuously until the edible wax toughens from chewed moisture and the flavor is finally dispersed. The edible wax eventually folds over itself, engulfing the container 300 into a chewed nub, that may then be discarded by the user.

In lieu of seep holes 332 or seep slits 330, container 300 can include cracks, capillaries, fissures, or any suitable conduit configured to transfer the confection from inside the cavity 340 to outside the container 300. Indeed, container 300 may be formed from a porous material capable of releasing the confection via a seeping or leaching action. Alternatively, container 300 can be formed from an absorbent material such as a sponge material or a foam material. In lieu of a confection contained in a cavity, the absorbent material may be impregnated with a confection that is released as the user chews the apparatus 100.

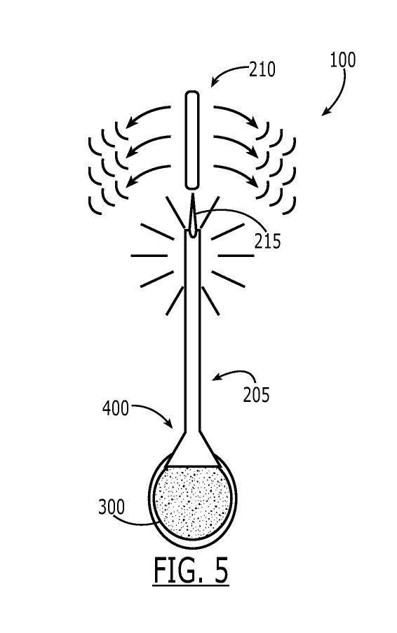

FIG. 4 is a schematic view of a portion of the confection delivery apparatus, in accordance with many embodiments. The number of seep holes or seep slits on the container 300 may vary in quantity and size in order to control the rate of product feed. In certain embodiments, the apparatus 100 may contain one to ten seep holes or slits varying in diameter or length. For instance, two seep holes 333 provide a slow product feed while four seep holes 334 provide for a faster feed, and eight seep holes provide for even faster feed. Accordingly, slow release would be controlled by smaller or fewer seep holes or slits. The contents of the cavity 340 are not limited to confections and may be, for example, used for slow release medicine or vitamins.

Since the container 300 is exposed to maximum bite force, the container 300 is flexible. The container 300 is hollow and comes in various sizes depending on the confection inside the cavity 340 which may include a hard shelled chocolate covered mint candy, a chocolate covered coffee bean, a chocolate covered mint soft shell candy, and any substance that is edible that may be contained in the container 300. The interior wall of the container 300 will vary in size depending on the contents of the cavity 340. The thickness of edible wax cocoon 310 will also depend on the contents of cavity 340. The stem 200, container 300, and joint 400 is formed of a material capable of withstanding external bite forces without breaking apart the apparatus 100.

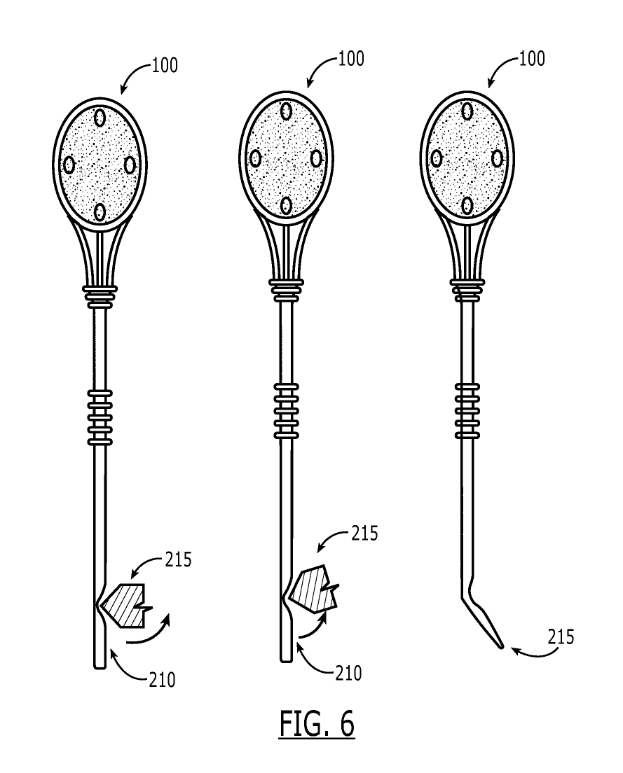

FIG. 5 is a schematic view of the confection delivery apparatus, in accordance with many embodiments. The stem 200 is flexible and can be round, square, triangle, flat, oval, or any suitable configuration. The second end 210 may be blunt shaped, square shaped, rounded like a ball, sharp and pointed, or any suitable configuration. At the first end 205 of the stem 200, flexible joint 410 includes a set of concentric rings (see FIG. 1) that act as a bending point. The flexible joint 410 functions as a pivot, allowing the stem 200 to bend in any direction without breaking After pivot point, the joint 400 thickens and flares out to form a positive attachment to container 300.

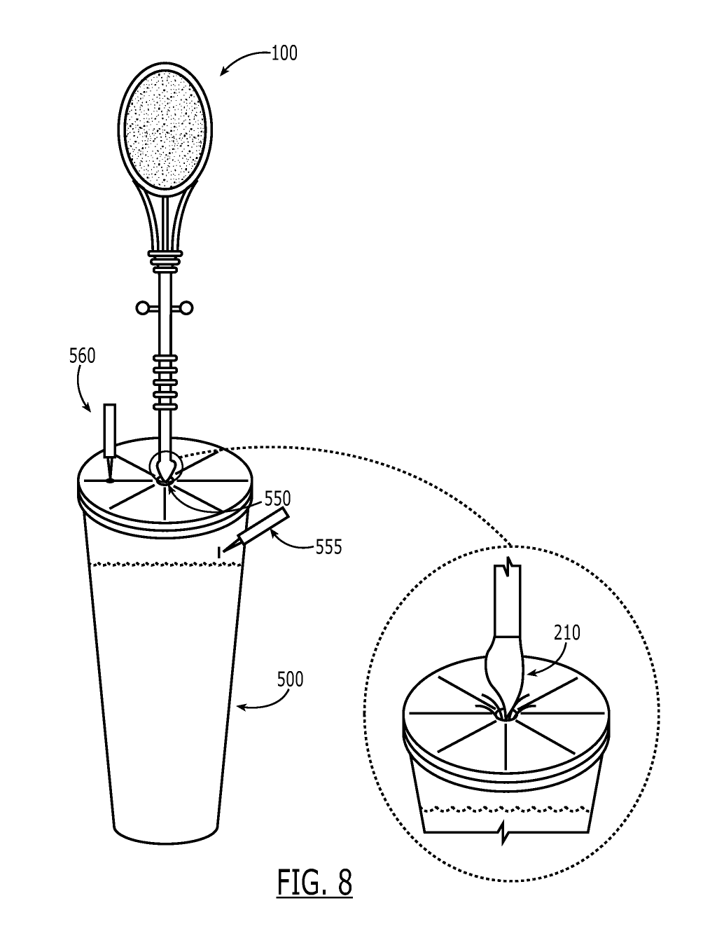

Between the second end 210 and the first end 205 is provided an intermediary portion 215 providing another pivot point. The thickness of the intermediary portion 215 is reduced compared to other portions of the stem 200 such that the intermediary portion 215 is flexible enough to bend without breaking This allows, for example, the intermediary portion 215 to bend the stem 200 into a J shape or L shape, and will be further described in FIG. 7

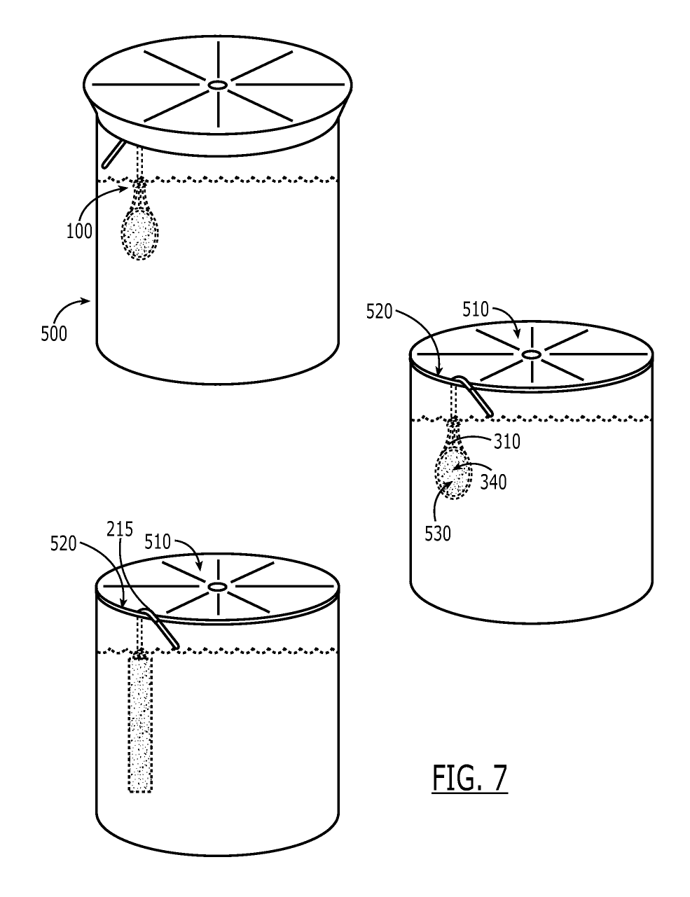

FIG. 6 is a schematic view of the confection delivery apparatus, in accordance with many embodiments. In FIG. 6, the intermediary portion 215 reduce in thickness gradually to form a thin portion that attaches to the thicker second end 210. A user may bend the second end 210 so as to break off the second end 210 from the apparatus 100. The intermediary portion 215 may also form a sharp incline that also allows the second end 210 to be broken off from the rest of the stem 200. Also shown in FIG. 6, the intermediary portion 215 may form a sharp point and be bent to form a curve with respect to the rest of the stem 200.

FIG. 7 is a cross-sectional view of the confection delivery apparatus and cup, in accordance with many embodiments. The confection delivery apparatus 100 is physically paired with a cup 500 filled with a drink, such as coffee. The cup 500 may include a lid 510. The J shaped stem 200 of apparatus 100 as described above is easily placed over a cup edge 520 to allow the container 300 to be submerged 530 in the drink of the cup 500 at a predetermined depth. In certain embodiments, the cavity 340 stores a chocolate covered espresso or regular coffee bean. Other flavors can be added such as mint, vanilla, and the like. The container 300 is dipped in a high temperature edible wax to form edible wax cocoon 310. The edible wax used may be a beeswax. The intermediary portion 215 may be a short, flexible flat portion approximately ½″ long to ensure that the apparatus 100 can be securely hung from the cup 500. The intermediary portion 215 is thin enough to allow the cup lid 510 to be placed over the lid 510 to secure the partially submerged apparatus 100 in the drink.

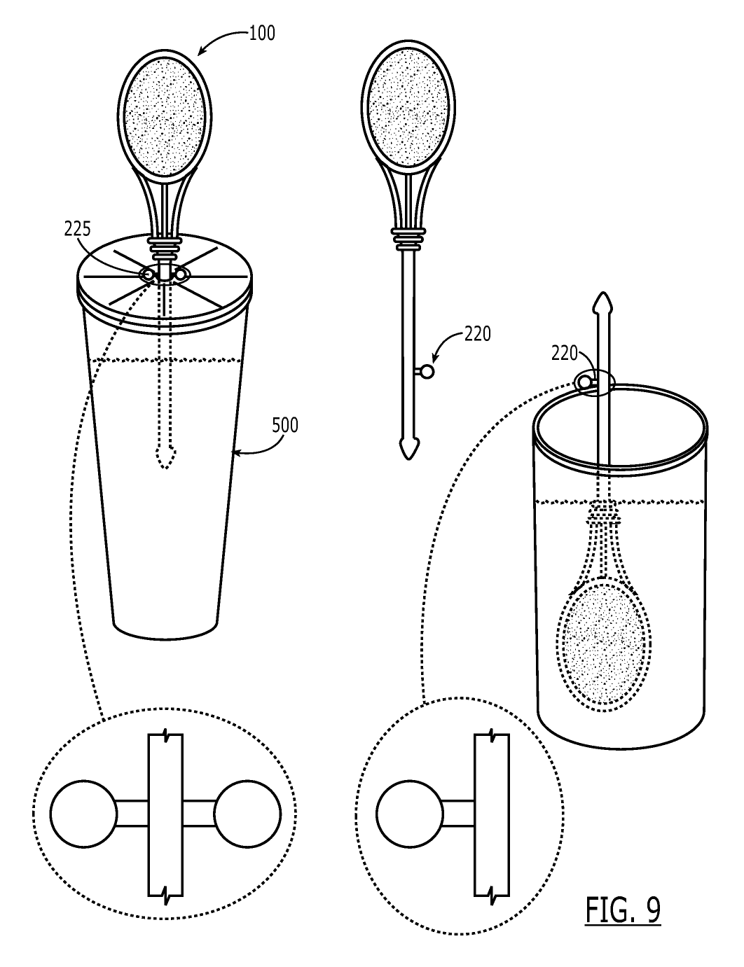

FIG. 8 is a cross-sectional view of the confection delivery apparatus and cup, in accordance with many embodiments. The second end 210 of the stem 200 forms a sharp flared point. The stem 200 is designed to penetrate the lid of cup 500 at any number of points 550, 555, 560, etc. This allows, for instance, the user to pair the apparatus 100 with cup 500 to use at any time while drinking from the cup 500. A harder edible wax is preferable for an apparatus 100 paired with a cup of coffee.

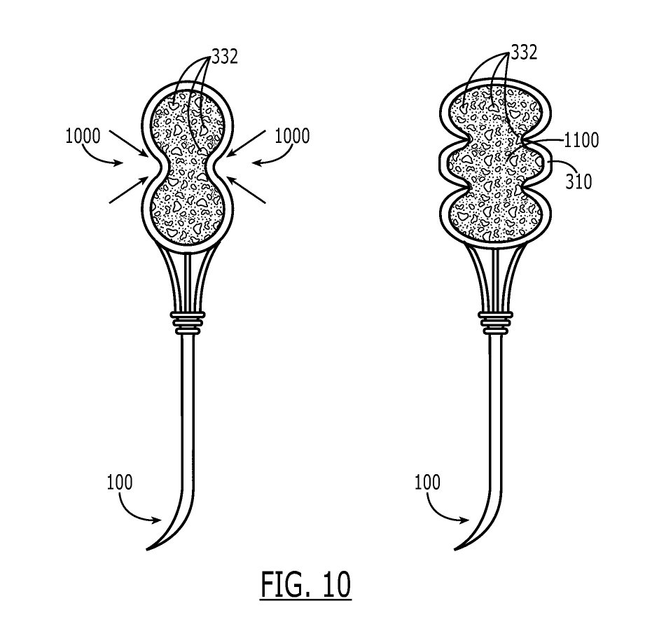

FIG. 9 is a cross-sectional view of the confection delivery apparatus and cup, in accordance with many embodiments. The stem 200 may include a single hanger protrusion 220 or a double hanger protrusion 225 that gauges how far the stem 200 will submerge inside the cup 500. The apparatus 100 may be hung from the cup lid or cup edge with the container 300 either submerged or above the drink. The protrusions may serve as a depth monitor as well.

The apparatus 100 beneficially provides a variable rate of release of the contents of the container 300 as a chewed edible wax receptor. The apparatus 100 further extends the taste of the stored confection considerably over time by mixing the chewed edible wax with mouth fluids such that the edible wax keeps trapping, folding and releasing flavor as the edible wax is chewed, extending the release of flavor. The chewable apparatus 100 creates a unique way to deliver confections in a time controlled delivery system.

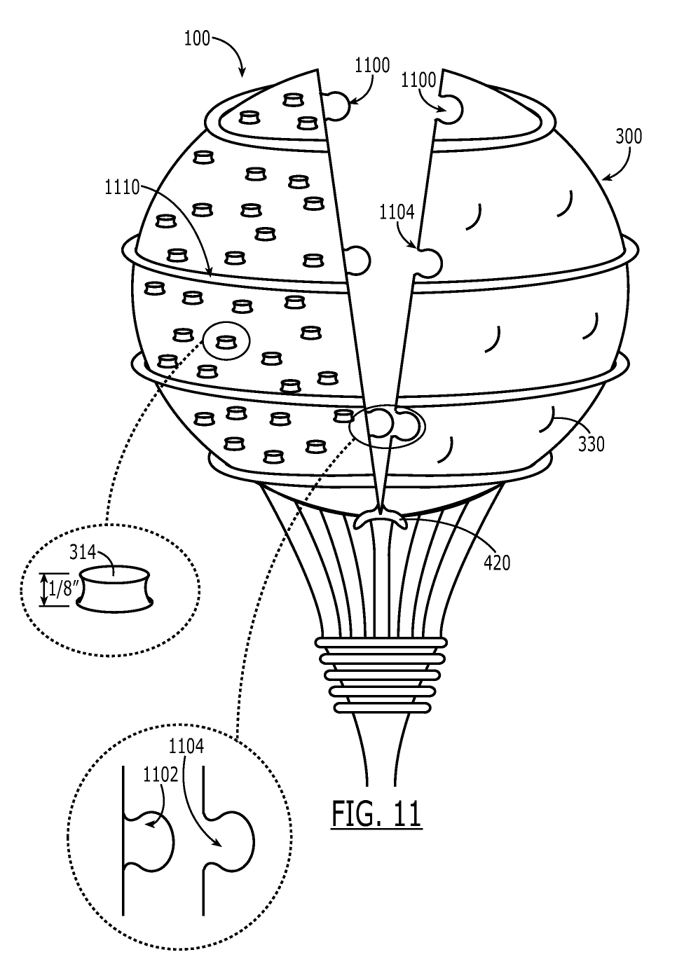

FIG. 10 is a cross-sectional view of a confection delivery apparatus, in accordance with many embodiments. The apparatus 100 is illustrated with the edible wax cocoon 310 and container 300 being compressed in response to an external force 1000, such as a biting force. The application of external force 1000 forces the confection within the cavity 340 to flow out of seep holes 332 and form a mixture 1100 of the edible wax and confection. The mixture 1100 will also include saliva if the external force 1000 is bite pressure. The mixture 1100 traps the confection in the edible wax and slowly releases the flavor as the mixture 1100 is chewed. The stem may be a flavored and form a tooth pick so as to remove food particles from teeth. The container 300 is preferably not resilient such that upon application of an external force on the container 300, the container retain its crushed shape. This advantageously allows the container 300 to be compressed continually be external force until all the contents of the cavity 340 are forced out through the conduits in the container 300 and are captured by the folding edible wax being chewed.

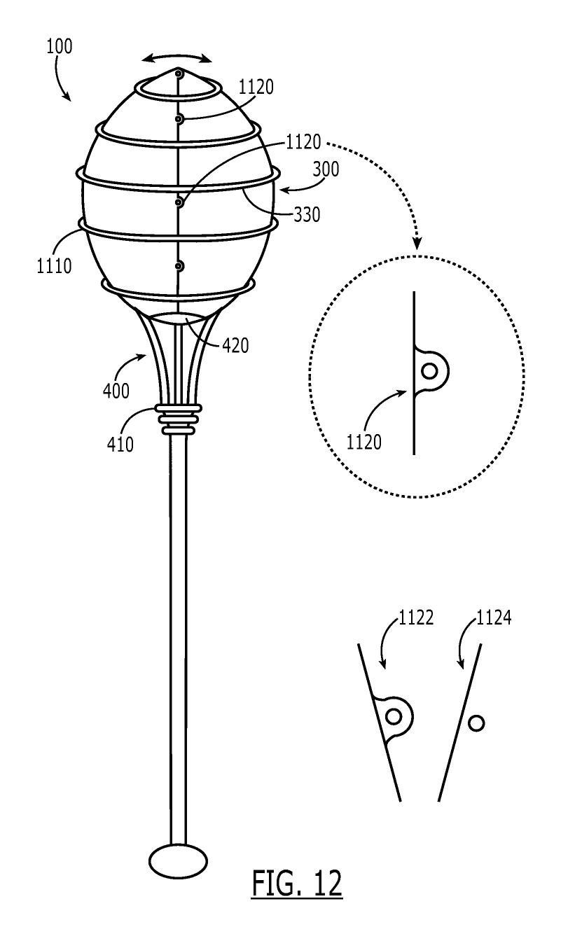

FIG. 11 is a detailed schematic view of a confection delivery apparatus, in accordance with many embodiments. FIG. 11 provides details on the configuration of the apparatus 100 to allow loading of product into the cavity 340 of the container 300. As shown in FIG. 11, the container 300 is provided in two portions that may be separated to expose the cavity 340 and allow the container 300 to be filled with a confection or other product. A first portion of the container 300 includes a set of projections 1102 and a corresponding set of recesses 1104 that form the interlock 1100. The projection 1102 engages with the corresponding recess 1104 by overlapping and/or by the fitting together to snap together and hold the container 300 together to secure the confection within the cavity 340 after loading of the confection in the cavity 340. The container 300 may include, for example, six or eight interlocks. The interlocks 1100 may be bendable to allow them to snap into place. Optionally, while the interlock 1100 may snap together or the like, the interlock 1100 may also comprise a plastic weld that also seals the contents of the container 300.

The joint 400 includes a reinforced portion 420 where the different portions of the container 300 meet to support the bending and insulation of the container 300 and confection. The reinforced portion 420 may be a thick bendable anchor that functions as a pivot or hinge for the container 300 and may include a thickness of ⅛″- 3/16″ and have a round, square or rectangle shape.

Furthermore, the wax anchors of the container 300 may further include a set of concentric rings 1110 that provide additional surface area to hold the edible wax cocoon 310, and may be ⅛″- 1/16″ in height. The wax anchors may also include a hollow protrusion 314 that may protrude, for example, ⅛″- 1/16″ from the container 300. The apparatus 100 of FIG. 11 includes a plurality of seep slits 330. The container 300 is loaded with, for example, a chocolate covered espresso coffee bean.

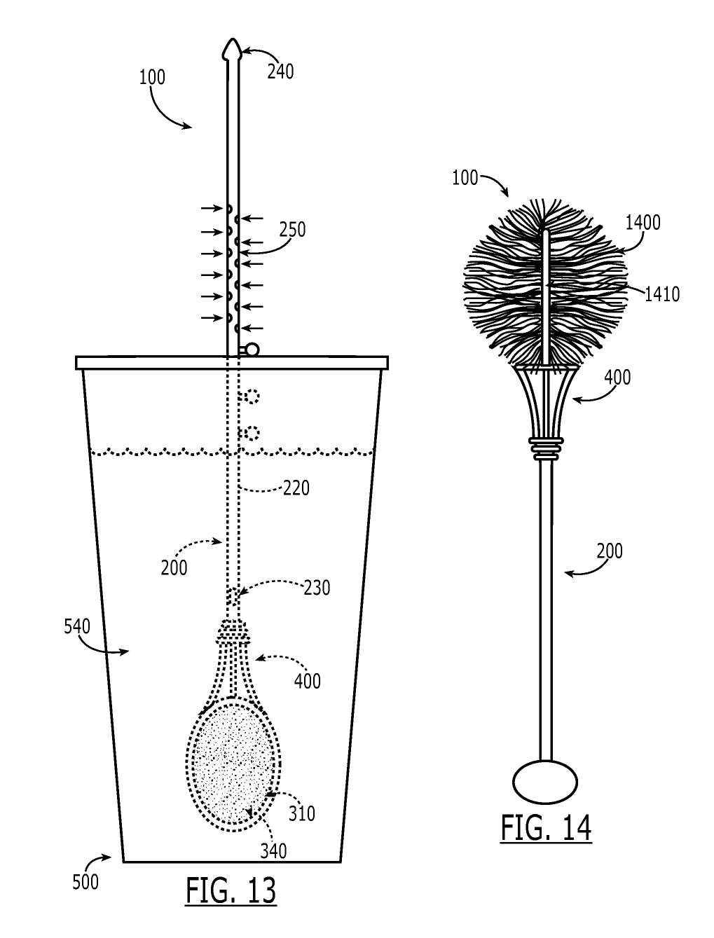

FIG. 12 is a schematic view of a confection delivery apparatus, in accordance with many embodiments. FIG. 12 illustrates a container 300 that has been snapped together by the interlock 1120. FIG. 12 illustrates a second type of snapping interlock 1120 including a projection with a buttonhole 1122 and a corresponding button 1124 that snap together.

The apparatus 100 may be loaded and coated as follows. The opened container 300 is first loaded with a confection and then the interlocks 1100 are snapped together. The apparatus 100 is cooled and then dipped in hot edible wax to form the airtight seal where the hot edible wax adheres to the surface of the container 300, including any wax anchors, rings, etc. that promote adhesion of the edible wax to the surface of the container 300.

In some embodiments, after the container 300 is loaded, interlocked and cooled, the apparatus 100 is dipped in very hot chocolate (e.g., dark or regular pure chocolate), then cooled again and thereafter dipped in hot edible wax to form the airtight seal. If the confection itself is provided a chocolate coating prior to loading, then the apparatus is provided two layers of chocolate coating to provide an extra shot of chocolate for an extra flavor rush. In other words, the confection is provided a chocolate coating within the cavity of the container, and a second chocolate coating layer is provided covering the container 300. Again, the edible wax cocoon may be infused with a complementary flavor prior to coating the container 300.

In FIG. 11, if the confection is chocolate covered, then a second chocolate coating layer would adhere to the surface of the container 300 and the hollow protrusions would provide additional surface area for the second chocolate coating layer to adhere so as to provide an additional flavor rush. In this manner, the apparatus 100 is double dipped in chocolate. An edible wax coating may then be applied to the second chocolate coating layer. The second coating layer need not be chocolate and may be any type of melted confection that can coat the outer surface of the container 300.

In some embodiments, the container 300 is dipped into any melted flavored confection that compliments the loaded confection. The apparatus 100 is then cooled before dipping in a chewy, hot, edible wax as a coating over the first coating. Preparation of the apparatus 100 in this manner would add additional tastes that would compliment the contents of the container 300. In this case, the wax anchors would be coated by the dipped chocolate or flavored substance. For instance, the hollow protrusions 314 would be filled with chocolate, adding an extra chocolate rush. The edible wax coating may also be mixed with a flavored substance to add additional flavor and/or contrast.

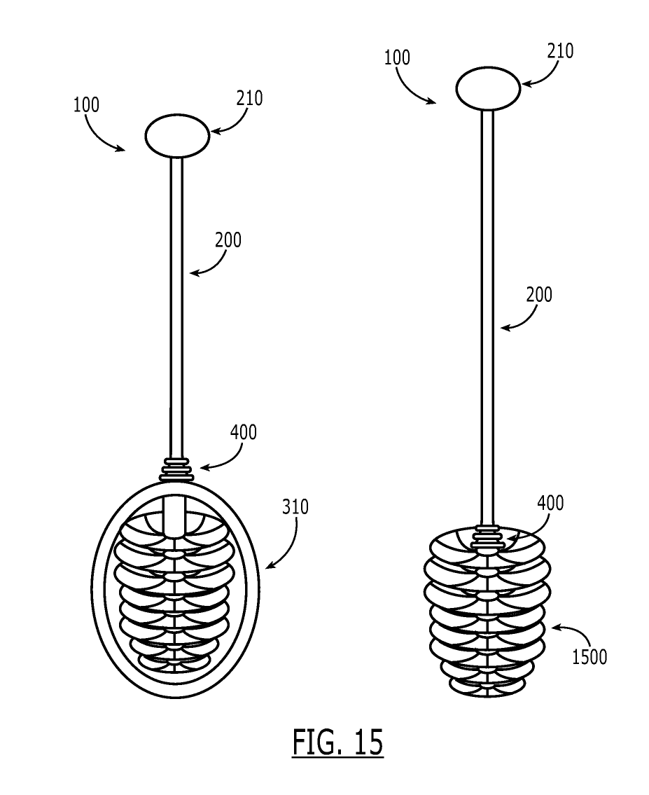

FIG. 13 is a cross-sectional view of a confection delivery apparatus and a cup, in accordance with many embodiments. The confection delivery apparatus 100 is physically paired with a cup 500 filled with a liquid 540, such as coffee. The stem 200 of apparatus 100 is illustrated with a plurality of hanger protrusions 220 placed along different portions of the stem 200 to allow the apparatus 100 to be hung from an edge of the cup 500 at different heights. In other embodiments, the hangers 220 are provided to hang the apparatus 100 from the same height for different sized cups. The apparatus 100 includes a cavity 340 and edible wax cocoon 310 as described above and may be submerged within a liquid 540 in the cup 500. The stem 200 includes a first conduit 230 adjacent to the joint 400 that allows the liquid 540 to enter a hollow passage 240 of the stem 200. The hollow passage 240 may extend throughout the entire stem 200 or just a portion of the stem 200. The first conduit 230 is illustrated as a single hole but may define a plurality of slits and holes adjacent the joint 400. A second conduit 250 is provided at another portion of the stem 200 opposite the joint 400. The hanger protrusion 220 may be provided between the first conduit 230 and second conduit 250 so that liquid flows through the first conduit 230 and external air flows through the second conduit 250. The end of the stem 200 opposite the joint 400 may be open or closed. In FIG. 13, the conduit 250 is illustrated as a plurality of holes.

Under this configuration, the apparatus 100 is submerged in the cup 500 from a hanger 220 such that hot coffee 540 flows into the hollow passage 240 via the first conduit 230. As the hot coffee fills the stem 200, cooler external air enters through second conduit 250 to aerate and cool the hot drink. The cooler air and hot coffee mix together to cool the coffee and allow a user to drink their coffee easily at a comfortable temperature more quickly. The number and size of the conduit 250 may be modified to control the amount of aeration provided. The thickness of the stem 200 with a hollow passage 240 may be modified to be stiff or pliable.

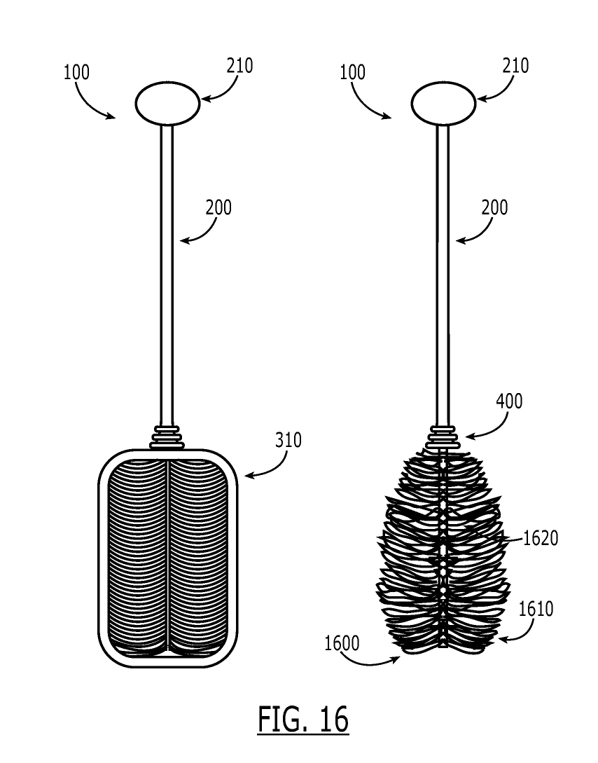

FIG. 14 is a schematic view of a confection delivery apparatus, in accordance with many embodiments. An apparatus 100 includes a stem 200 and a flexible joint 400 connected thereto to join the stem 200 to branches 1400 that protrude outward. The spaces between the branches 1400 may be filled by dipping and/or coating. The joint 400 may be a thick bendable anchor that functions as a pivot or hinge for the branches 1400. The branches include a set of thin collapsible brush strands protruding outward from a bendable anchor 1410 thinner than the stem 200. The bendable anchor 1410 protrudes from the stem 200. The components of the apparatus 100 may be all of the same material, but varied in strength, flexibility and resiliency based upon a thickness and/or shape of the component.

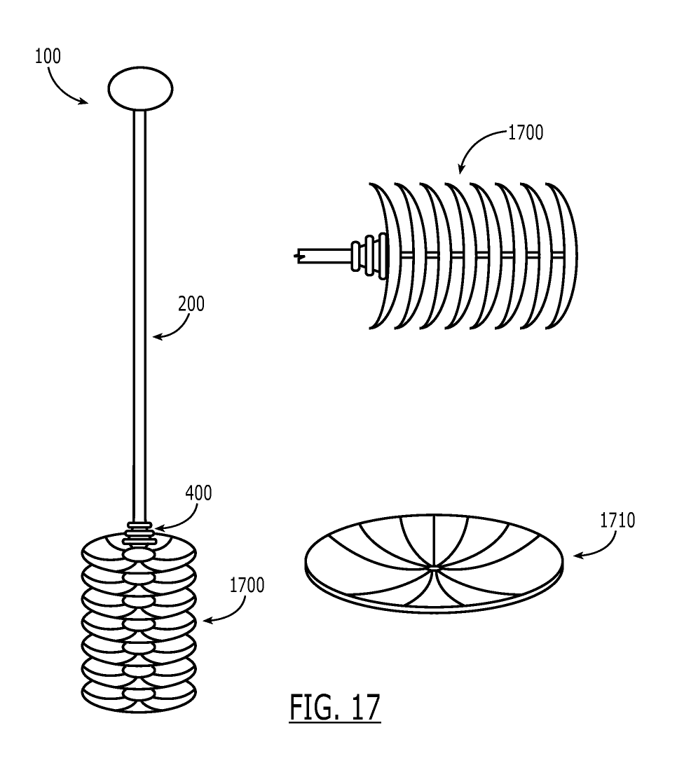

FIG. 15 provides schematic views of a confection delivery apparatus, in accordance with many embodiments. The apparatus 100 of FIG. 15 includes a solid flexible stem 200 that may include a second end 210 in the shape of a knob, a bendable joint 400 for connecting the stem 200 to baffles 1500. The baffles 1500 include a set of upturned cupped collapsible baffles. When dipped in a liquid substance such as a confection, the substance is trapped in between the curves of the baffles 1500. After curing, the baffles 1500 may be dipped again to fill in any voids left after the first dipping with the same or different substance, such as a different confection. The apparatus 100 may be dipped in an edible wax to create a sealed wax cocoon 310 for chewing.

The baffles 1500 are preferably formed by injection molding of flexible plastic or other suitable material. As described herein, the baffles turned upward refers to the baffles curving towards the stem 200. The baffles 1500 may be provided in a plurality of shapes such as round, oval, oblong, square, star, diamond, rectangle, triangle, etc., so long as the baffles are stackable, upturned and collapsible. The confection in between the baffles may include layers of mint and dark chocolate, for example. The upturned baffles 1500 are crushable when compressed. In this configuration, the baffles fold under external pressure, such as a bite force, and trap flavors to extend the flavor rush. Continual chewing may allow the user to chew out all the flavor from the baffles.

The baffles are preferably formed from a pliable material that compresses when chewed. The baffles 1500 may be thinner than the stem 200 so as to allow the baffles 1500 to compress and remain collapsed in response to an application of external force. The baffles initially hold an uncompressed shape and compresses upon itself in response to the application of an external force (such as a chewing force) and retains the compressed shape in response to removal of the external force. Baffles 1500 may be formed from a non-resilient material that does not return to its original shape when compressed by an external force. The baffles 1500 may be formed from any suitable plastic, rubber, nylon, composite or other material. However, the hardness, resiliency, elasticity, tensile strength and other physical properties of the baffles 1500 may be selected in accordance with the confection and the application of a large external compressing force such as a bite force. The surfaces of the baffles may be smooth, rough or ribbed.

The baffles may be formed via a one piece injection mold. The baffles may be formed of the same plastic material as the rest of the apparatus, just thinner. The thickness and size of the baffles will vary depending on the consistency of the confection used and application. The length of a baffle anchor may be approximately 1.00-1.25 inch in length and approximately 0.50-0.75 inch in width, for example. The depth of the baffle may be approximately 0.0625-0.25 inches for a cupped baffle. The baffles may be placed approximately 0.0625-0.125 inches apart from each other and the apparatus may include 4-8 cupped baffles, for example. Baffles having a cupped shape will retain more of the confection compared to a flat baffle.

In addition, a cupped shaped baffle has the further advantage that when a bite force is applied, the baffle will naturally fold into itself, forcing the filled confection inward and upward, providing a mixing effect that blends confection flavors together. The chewy wax cocoon will start folding over the flavors to extend the overall flavor experience. As wax is chewed and flavors are consumed, the wax becomes harder from mouth liquids and soon folds over the spent baffles to create a hard nub that is easily disposed of by a user.

The wax cocoon may be replaced by a high temperature confection coating that forms a hard shell around the dipped confection(s) using any flavor and softness/hardness. Alternatively, the apparatus may be provided without a wax cocoon or a hard shell confection. For example, baffles may be dipped into a banana confection and dipped in vanilla cream confection, and then dipped one to multiple times in dark chocolate and then cooled and packaged as a single confection treat.

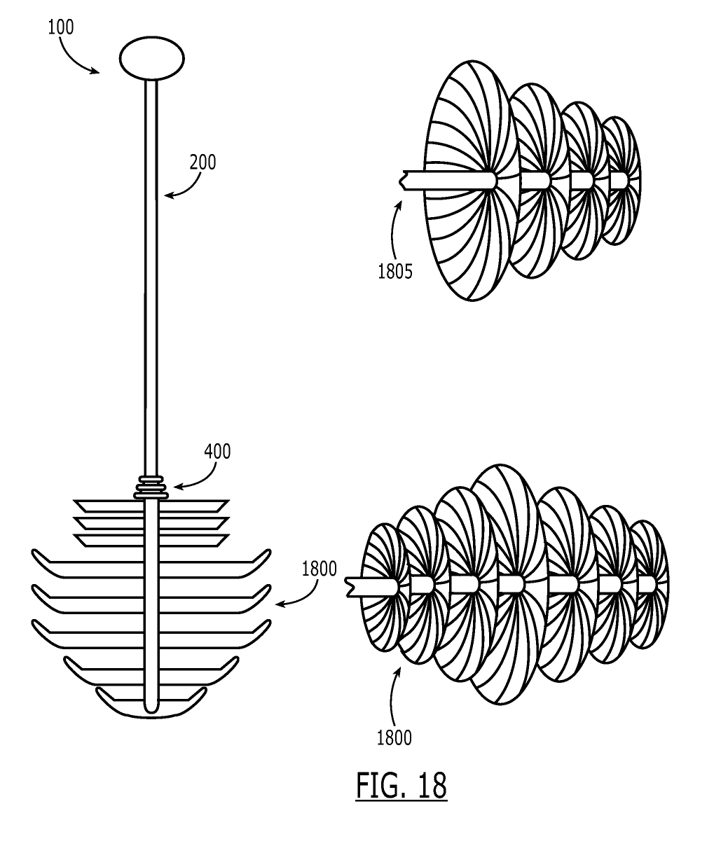

FIG. 16 provides schematic views of a confection delivery apparatus, in accordance with many embodiments. The apparatus 100 in FIG. 16 includes a solid bendable stem 200 connected to a flexible joint 400 which in turn is connected to branches 1600 including a series of thin collapsible brush strands 1610 protruding from a thin bendable anchor 1620. The strands 1610 curl upward towards the stem 200.

A non-limiting example of a dipped confection delivery apparatus includes the apparatus 100 of FIG. 16 dipped in a melted edible flavored mint substance. The heated confection is then cooled so that the confection clings to the thin fine strands 1610, which then may be re-dipped into dark melted chocolate or any flavored substance. After cooling briefly, the apparatus 100 is then dipped into an edible wax that may be laced with a flavor to complement the other flavors. The edible wax cools to form a wax cocoon 310 to seal the flavors together. The apparatus 100 may be formed by injection molding with flexible plastic.

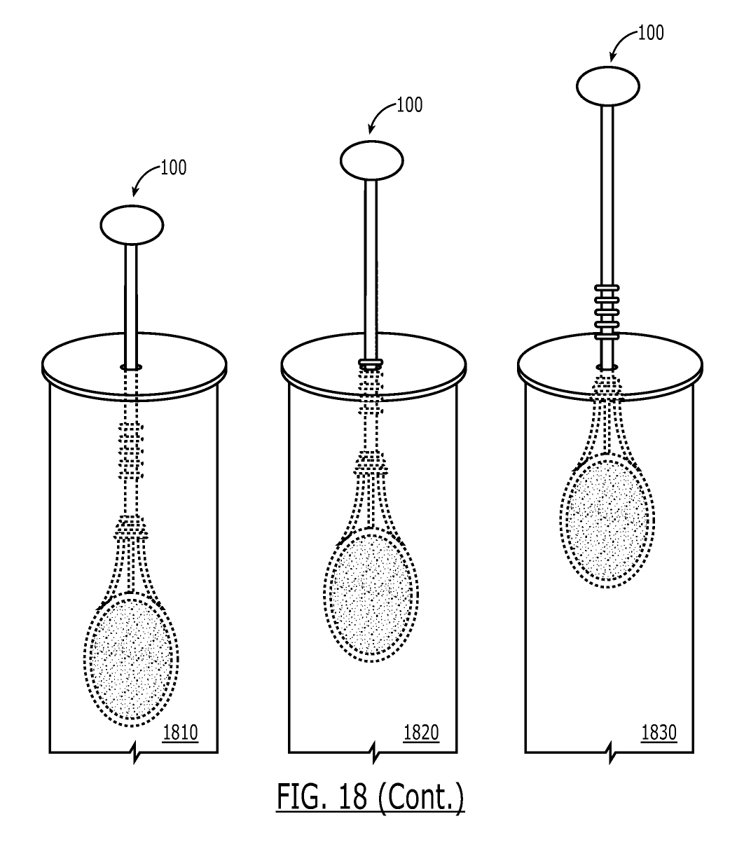

FIG. 17 provides schematic views of a confection delivery apparatus, in accordance with many embodiments. The apparatus 100 includes a stem 200, joint 400 and baffles 1700. The baffles 1700 curve upward toward the stem 200 and include a plurality of baffles spaced apart to allow a liquid substance to fill the void between the baffles and cure. The baffles 1700 are injection moldable. The baffles are preferably provided in parallel to each other and perpendicular to the stem. A baffle 1710 may generally be cupped to provide a volume to hold the confection. The baffle 1710 is also collapsible in response to an external pressure, such as a user bite force. The baffles are preferably not resilient such that upon application of an external force, the baffles retain their crushed shape. This advantageously allows the baffles to be compressed continually by external force until all the contents of the baffles are forced out and are captured by the folding edible wax being chewed.

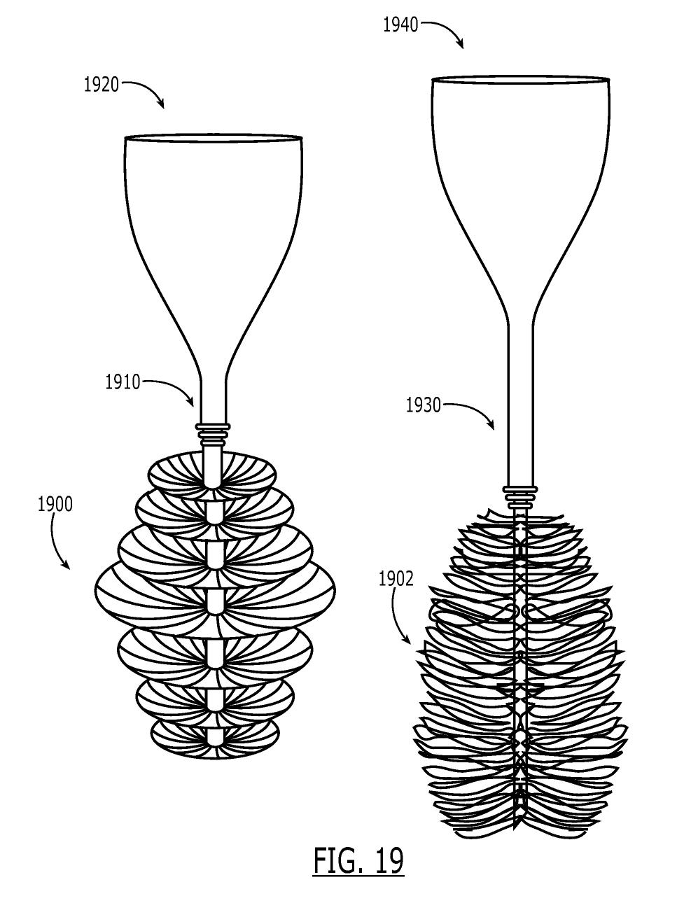

FIG. 18 provides perspective views of a confection delivery apparatus 100, in accordance with many embodiments. The baffles 1800 may be upturned towards the stem 200 near the edges of each baffle to hold in a dipped substance within the baffle. The baffles are attached together by a flexible stem 1805 that protrudes from joint 400 and stem 200. The diameter of the baffles 1800 may be varied.

The apparatus 100 may be first dipped into a melted first confection 1810 and cooled. Then, apparatus 100 may be dipped a second time in a melted second confection 1820. This dipping and cooling may be repeated as many times as desired to form a desired flavor and texture combination. The final layer is formed by dipping the apparatus in edible wax 1830 to form the wax cocoon.

In another embodiment, the apparatus may be first cooled by freezing or near freezing such that the flexible baffles will stiffen. Then, the apparatus may be dipped into a confection where the frozen cupped baffle and stem will almost instantly begin cooling the soft dipped confection. The use of a cold apparatus for loading a confection advantageously improves dipping speeds and allows for higher production.

For example, an apparatus may be dipped in a confection to cover half of the cold baffles with one flavor. After the dipped confection is cooled by the cold baffles, the apparatus is re-dipped in a complementary confection to cover the remaining undipped baffles and cooled as well. As the two confections are cooled, the apparatus may be dipped into a plain or flavored (e.g., vanilla, coconut) edible chewy wax to form an airtight seal.

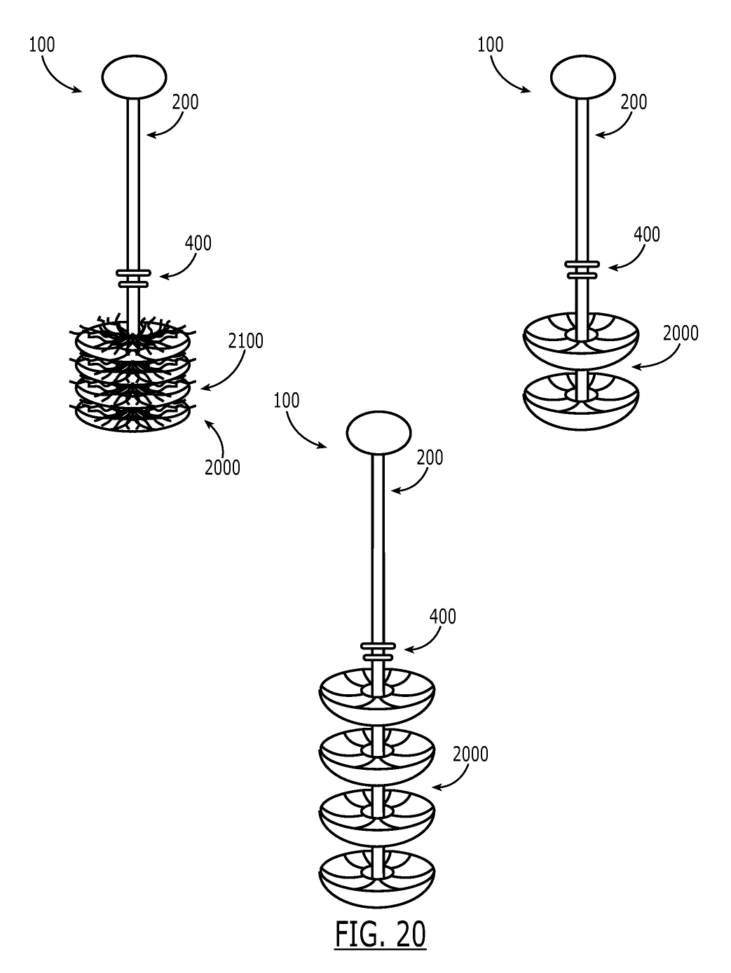

FIG. 19 provides schematic views of a confection delivery apparatus, in accordance with many embodiments. Apparatuses may be provided with a baffled head 1900 and a branched head 1902. The heads 1900, 1902 are connected to respective stems by a flexible joint. FIG. 19 illustrates a short stem 1910 including a flat grip handle 1920 that allows a user to easily grip and handle the confection delivery apparatus. The head 1902 is connected to a stem 1930 longer than that of short stem 1910. The end of stem 1930 includes a flat handle grip 1940 that may provide sufficient surface area for a user to grip and control the confection delivery apparatus.

The confection delivery apparatuses described herein may be placed in low-temperature storage in order to considerably extend the shelf life of the apparatus. The apparatuses 100 described herein may be dipped in only one flavor, and then sealed with an unflavored edible wax cocoon 310. The apparatus 100 may also be half dipped in one substance (e.g., mint), and then cured before being fully dipped in a second substance (e.g., dark chocolate) to create a half-mint, half-chocolate mix on a bottom portion of the apparatus and solid chocolate on a top half of the apparatus. The apparatus 100 may be further dipped in a vanilla laced edible wax to create a wax cocoon.

The apparatus 100 may be used to form a variety of different flavors and textures using different combinations of confections that are sealed airtight within an edible wax cocoon that itself may be flavored. The sealed apparatus 100 is ready to store and eat and creates a flavor extension that is immediately ready for consumption. The apparatus 100 may be dipped three, four or a plurality of times in different substances to complement each of the flavors. Confections may be used individually or layered in combination and include breath mints, dessert mints with chocolate, novelty items such as banana cream pie and coconut cream pie.

The edible wax cocoon serves to form an airtight seal of the confections, and may be laced with a flavor itself, such as coconut, vanilla or any flavor that complements the flavors within the wax cocoon. The chewy wax cocoon further acts as a confection flavor delivery system in that when the apparatus 100 is chewed, all the dipped flavors are mixed and crushed together by the external pressure applied. Portions of the chewed confection becomes folded into the chewed wax cocoon and acts to extend the flavor burst over a longer period of time, thereby creating a more satisfying taste experience without the mess of conventional soft confection products. When the baffles and edible wax have been depleted of flavor, the user applying the external bite force chews the baffles and wax into a nub that is discarded without mess on the hands or face.

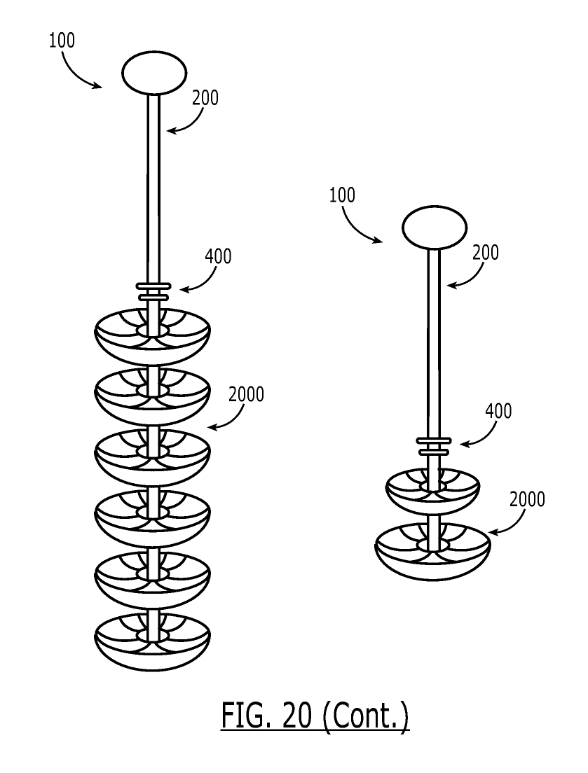

FIG. 20 provides schematic views of a confection delivery apparatus, in accordance with many embodiments. FIG. 20 illustrates a variety of apparatuses 100 in different configurations. The apparatus 100 may include a head including a stem that alternates between the brush strands 2100 and baffle cup 2000. Other embodiments include apparatuses with different numbers of stacked baffles such as 2, 4 or 6. The spacing between baffles may be varied, as well as the size of each baffle within an apparatus may be varied in order to highlight or complement the confection flavors in different ways.

The embodiments described in FIGS. 14-20 may also be dipped into a cup of liquid, such as a cup of hot coffee in order to complement the overall experience by heating and softening the confection within the interior of the wax cocoon without creating a mess.

The stems described in FIGS. 14-20 may include a hollow passage that may extend throughout the entire stem or a portion of the stem 200 as described and illustrated in FIG. 13. The end of the stem 200 opposite the joint 400 may be open or closed and the stem may include a plurality of holes. In this manner, the baffle head or brush head may be attached to a stem having a hollow passage for allowing liquid and air to flow through the stem to provide a cooling effect when the apparatus is dipped into a hot cup of coffee, for example.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

Accordingly, the specification and figures associated with these embodiments are to be regarded in an illustrative rather than a restrictive sense, and all modifications are intended to be included within the scope of the claims described below. The benefits, advantages, solutions to problems, and any element(s) that may cause any benefit, advantage, or solution to occur or become more pronounced are not to be construed as a critical, required, or essential features or elements of any or all the claims.

1. A confection delivery apparatus, comprising:
a chewable set of spaced apart baffles formed from a pliable and non-resilient material;
said baffles connected perpendicular to an anchor;
a joint connected to said anchor;
a stem connected to said joint;
said baffles curving toward said stem;
said space between said baffles including a confection; and
an edible wax coating adhered to said baffles and said confection.

2. The apparatus of claim 1, wherein said anchor is thinner than said joint.

3. The apparatus of claim 1, wherein said baffles are thinner than said stem.

4. The apparatus of claim 1, wherein said baffles define at least one of a smooth, rough and ribbed surface.

5. The apparatus of claim 1, wherein said baffles are formed by injection molding.

6. The apparatus of claim 1, wherein said baffles define at least one of an ellipsoid and a polygon shape.

7. The apparatus of claim 1, wherein said baffles vary in diameter.

8. The apparatus of claim 1, wherein said stem defines at least one hanger.

9. The apparatus of claim 1, wherein said edible wax coating temporarily seals said confection within said baffles.

10. A confection delivery apparatus, comprising:
a chewable set of spaced apart branches formed from a pliable and non-resilient material;
said branches connected to an anchor;
a joint connected to said anchor;
a stem connected to said joint;
said branches curving toward said stem;
said space between said branches including a confection; and
an edible wax coating adhered to said branches and said confection.

11. The apparatus of claim 10, wherein said anchor is thinner than said stem.

12. The apparatus of claim 10, wherein said branches are thin collapsible brush strands.

13. The apparatus of claim 10, wherein said branches and said anchor are formed by injection molding.

14. The apparatus of claim 10, wherein said branches vary in length.

15. The apparatus of claim 10, wherein a first portion of said branches are covered by a first confection and a second portion of said branches are covered by a second confection.

16. The apparatus of claim 10, wherein said edible wax is laced with a flavoring.

17. The apparatus of claim 10, wherein said stem defines a first end contacting said joint, a second end opposite said first end, wherein said second end defines a flat handle.

18. The apparatus of claim 10, wherein the confection includes any of a breath mint, dessert mint, banana cream pie, and coconut cream pie.

19. The apparatus of claim 10, further comprising:
a cup of liquid attachable to said stem of said apparatus.

20. A method of forming a confection delivery apparatus, comprising the steps of:
cooling a chewable set of spaced apart baffles formed from a pliable and non-resilient material, said baffles connected perpendicular to an anchor, a joint connected to said anchor, a stem connected to said joint, said baffles curving toward said stem; loading a confection within the baffles;
coating said baffles with a melted confection;
cooling said melted confection via the cooled baffles so as to form a melted confection coating adhered to said baffles;
coating said melted confection coating of said confection delivery apparatus with a melted edible wax; and
cooling said melted edible wax so as to form an edible wax coating adhered to said melted confection coating, wherein the confection is forced through said baffles in response to an external force and remain compressed in response to removal of said external force.

21. The method of claim 20, further comprising:
freezing the confection delivery apparatus;
attaching the frozen confection delivery apparatus to a cup of liquid; and
cooling the liquid in the cup by submerging the frozen confection delivery apparatus in the liquid.

22. The method of claim 21, wherein the liquid is heated.

23. The method of claim 22, wherein the liquid is coffee.

24. A method of cooling a hot liquid, comprising:
providing a chewable set of spaced apart baffles formed from a pliable and non-resilient material, wherein said baffles are connected perpendicular to an anchor, a joint is connected to said anchor, a stem is connected to said joint, said baffles curve toward said stem, said space between said baffles includes a confection, and an edible wax coating is adhered to said baffles and said confection;
freezing the confection delivery apparatus;
attaching the frozen confection delivery apparatus to a cup of liquid;
cooling the liquid in the cup by submerging the frozen confection delivery apparatus in the liquid.

25. A method of cooling a hot liquid, comprising:
providing a chewable set of spaced apart baffles formed from a pliable and non-resilient material, wherein said baffles are connected perpendicular to an anchor, a joint is connected to said anchor, a stem is connected to said joint, said baffles curve toward said stem, said space between said baffles includes a confection, and an edible wax coating is adhered to said baffles and said confection;
freezing the confection delivery apparatus;
attaching the frozen confection delivery apparatus to a cup of liquid;
cooling the liquid in the cup by submerging the frozen confection delivery apparatus in the liquid.

26. The method of claim 25, wherein the frozen confection delivery apparatus is attached over an edge of the cup by a curved portion of the stem.

27. The method of claim 25, further comprising:
attaching a lid to the cup such that the frozen confection delivery apparatus is between the cup and the lid.

28. The method of claim 25, further comprising:
melting the confection delivery apparatus by heat of the liquid.
